(19) United States
(12) Patent Application Publication
(10) Pub. No.: US 2023/0265044 A1
(43) Pub. Date: Aug. 24, 2023

(54) SALT OF DEMETHYL TRAMADOL AND USE THEREOF

(71) Applicant: NANJING NORATECH PHARMACEUTICAL CO., LTD., Nanjing, Jiangsu (CN)

(72) Inventors: Fei LIU, Nanjing, Jiangsu (CN); Gang WU, Nanjing, Jiangsu (CN); Xiaobo WANG, Nanjing, Jiangsu (CN); Yehai XU, Nanjing, Jiangsu (CN); Lin WANG, Nanjing, Jiangsu (CN); Minqiang ZHENG, Nanjing, Jiangsu (CN)

(21) Appl. No.: 18/003,947

(22) PCT Filed: Jun. 25, 2021
(86) PCT No.: PCT/CN2021/102494

§ 371 Date: Dec. 30, 2022

(30) Foreign Application Priority Data
Jun. 30, 2020 (CN) 202010622340.3

(51) Int. Cl. C07C 215/64; C07C 65/11

(52) U.S. Cl. CPC C07C 215/64; C07C 65/11; C07C 2601/14

(57) ABSTRACT

The present invention provides a salt of demethyl tramadol, i.e., a compound represented by formula (I) or a solvate thereof, a pharmaceutical composition comprising the compound represented by formula (I) or the solvate thereof, and a use of the compound represented by formula (I) or the solvate thereof or the pharmaceutical composition in the treatment of moderate to severe pain. The compound or pharmaceutical composition of the present invention can stably release a drug having analgesic activity in a body for a long period of time, can be conveniently used by doctors and patients while exerting pharmacological effects, and has good medication compliance.

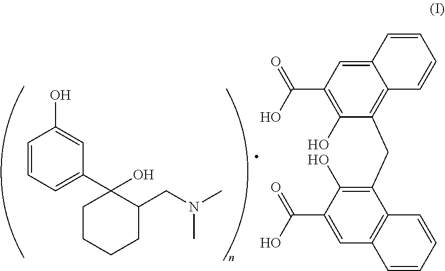

### TECHNICAL FIELD

The present invention relates to the field of pharmaceutical chemistry. In particular, the invention relates to an insoluble salt of demethyl tramadol, a preparation method for the insoluble salt, a pharmaceutical composition comprising the insoluble salt, and a medical use of the insoluble salt or pharmaceutical composition.

### BACKGROUND

Postoperative pain is an acute pain that occurs immediately after a surgery, the nature of which is an acute nociceptive pain, and it is also the acute pain which is the most common and needs urgent treatment in clinic. If not fully controlled in the initial state, it is readily to develop into a postoperative chronic pain. Opioids are commonly used in clinic to treat postoperative pain, but there are adverse reactions, such as respiratory depression and addiction. Local anesthetics are also the most important analgesic drugs in clinic, including lidocaine, bupivacaine, ropivacaine, and the like. Existing local anesthetics have relatively short effective time and need to be repeatedly administered. Therefore, it is urgent to develop analgesic and anesthetic drugs with small side effects and long-term efficacy.

Tramadol is a central synthetic opioid analgesic, the structure of which is related to codeine and morphine. Generally, tramadol is considered as a low-risk opioid for moderate to severe pain due to its good tolerance, such as low short-term addiction and low risk of respiratory depression. Tramadol is effective for various types of pain, including neuropathic pain, postoperative pain, fibromyalgia, osteoarthritis and cancer related pain. According to the Chinese Pharmacopoeia, the chemical name of tramadol hydrochloride is (±)-(1RS, 2RS)-2-[(N,N dimethylamino)methylene]-1-(3-methoxyphenyl)cyclohexanol hydrochloride.

After tramadol enters the human body, there are two main metabolic pathways, including generating O-demethyltramadol through O-demethylation catalyzed by CYP2D6 and generating N-demethyltramadol through N-demethylation catalyzed by CYP3A4. However, different patients will show different pharmacokinetic characteristics after taking tramadol due to the polymorphism in CYP2D6 gene, which will affect the enzymatic activity of CYP2D6. Patients with strong CYP2D6 enzyme activity will generate a high concentration of the active metabolite, O-demethyltramadol in their bodies, which may lead to a risk of tramadol-poisoning. While in patients with weak CYP2D6 enzyme activity, the concentration of active metabolite of tramadol is low, which will reduce analgesic effects. The development of long-acting preparations of tramadol may lead to individual differences in efficacy and even poisoning risks. As the active metabolite of tramadol, O-demethyltramadol exhibits higher activity than tramadol.

Therefore, there is an urgent need for a safe and effective drug with simple production process that can stably release the anesthetic drug in the body for a long time. The drug can not only maintain a continuous release for a long period of time, prolong analgesic effects, but also facilitate the use of such drugs by doctors and patients, thereby possessing good drug compliance.

### SUMMARY OF THE INVENTION

The purpose of the present invention is to provide an insoluble palmitate of demethyl tramadol or a solvate thereof.

Another purpose of the present invention is to provide a pharmaceutical composition comprising the insoluble salt or a solvate thereof.

The insoluble palmitate of demethyl tramadol or a solvate thereof and the pharmaceutical composition of the present invention can continuously release active demethyl tramadol in the body, thereby maintaining the drug concentration for a long time and achieving long-term analgesic effects.

In the first aspect, a compound of formula (I) or a solvate thereof is provided in the present invention

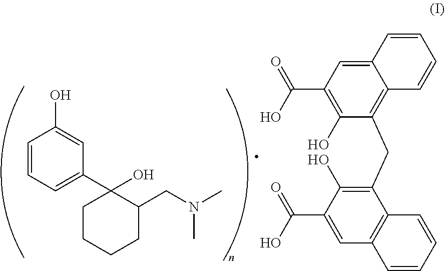

wherein n is an N is an integer from 1 to 4.

In a specific embodiment, n is 1 or 2.

In a specific embodiment, n is 2.

In a preferred embodiment, the solvate is a solvate formed by the compound of formula (I) and methanol, ethanol, n-propanol, isopropanol, ethyl acetate, acetone or water.

In a preferred embodiment, the D50 value of the compound of formula (I) or a solvate thereof is 0.1 to 100 μM.

In a preferred embodiment, the compound of formula (I) is a racemic palmitate of demethyl tramadol, and the chemical name of the racemic demethyl tramadol is (±)-(1RS, 2RS)-2-[(N,N dimethylamino)methylene]-1-hydroxy-cyclohexyl phenol.

In a preferred embodiment, the compound of formula (I) is a palmitate of dextral demethyl tramadol or palmitate of levo demethyl tramadol or palmitate of racemic demethyl tramadol.

In a preferred embodiment, the compound of formula (I) is a palmitate of dextral demethyl tramadol.

In a preferred embodiment, the solubility of the compound of formula (I) in water is not higher than 0.8 mg/mL; preferably, not higher than 0.7 mg/mL; and more preferably, not higher than 0.5 mg/mL.

In a preferred embodiment, the hygroscopicity of the compound of formula (I) is not higher than 7%; preferably, not higher than 4%; and more preferably, not higher than 3%.

In a preferred embodiment, the content of a single impurity of the compound of formula (I), after stored under a light condition (4500 lux±500 lux) for 10 days, is not higher than 0.1%; preferably, not higher than 0.06%; and more preferably, not higher than 0.04%; and the content of total impurities is not higher than 0.20%; preferably, not higher than 0.10%; and more preferably, not higher than 0.04%; and the content of a single impurity, after stored under a light condition (4500 lux 500 lux) for 30 days, is not higher than 0.15%; preferably, not higher than 0.10%; and more preferably, not higher than 0.05%; and the content of total impurities is not higher than 0.40%; preferably, not higher than 0.20%; and more preferably, not higher than 0.05%.

In a preferred embodiment, the content of a single impurity of the compound of formula (I), after stored under a high temperature and high humidity condition (40° C., 75% humidity) for 10 days, is not higher than 0.08%; preferably, not higher than 0.06%; and more preferably, not higher than 0.05%; and the content of total impurities is not higher than 0.20%; preferably, not higher than 0.10%; and more preferably, not higher than 0.05%; and the content of a single impurity, after stored under a high temperature and high humidity condition (40° C., 75% humidity) for 30 days, is not higher than 0.1%; preferably, not higher than 0.06%; and more preferably, not higher than 0.05%; and the content of total impurities is not higher than 0.30%; preferably, not higher than 0.20%; and more preferably, not higher than 0.05%.

In a preferred embodiment, the plasma half-life (t_{1/2}) of the compound of formula (I) is at least 16 hours; and preferably, at least 17 hours.

In a preferred embodiment, the maximum plasma concentration (Cₘₐₓ) of the compound of formula (I) is not higher than 500 ng/mL; and preferably not higher than 270 ng/mL.

In the second aspect, a pharmaceutical composition is provided in the present invention, which comprises the compound of formula (I) or solvates thereof, and optional pharmaceutically acceptable excipients.

In a preferred embodiment, the pharmaceutically acceptable excipients include but are not limited to one or more selected from the following group: a suspending agent, surfactant, filler, preservative, isotonic regulator, pH regulator, buffer and water.

In a preferred embodiment, the surfactant is polysorbate 20 (Tween-20).

In a preferred embodiment, the buffer is sodium dihydrogen phosphate.

In a preferred embodiment, the compound of formula (I) or solvates thereof is a solid particle with a D50 value of 0.1 to 100 μm.

In a specific embodiment, the pharmaceutical composition is a dosage form suitable for the subcutaneous, intradermal or intramuscular injection.

In a specific embodiment, the pharmaceutical composition is in a form of a depot formulation; and preferably, a long acting solid particle suspension injection.

In a specific embodiment, the pharmaceutical composition comprises 10 to 1000 mg; preferably, 50 to 200 mg of the compound or solvates thereof in 1 mL of the suspension injection.

In the third aspect, the use of the compound of formula (I) or solvates thereof of the first aspect, or the pharmaceutical composition of the second aspect is provided in the present invention, for preparing a medicament for preventing, treating or relieving pain.

In a preferred embodiment, the medicament for relieving pain is a medicament for moderate to severe pain.

In a preferred embodiment, the moderate to severe pain is a postoperative pain. In a preferred embodiment, the postoperative pain is an acute or chronic postoperative pain.

In a preferred embodiment, the medicament for relieving pain is a safe and effective anesthetic drug stably releasing active ingredients in vivo for a long time.

In a preferred embodiment, the medicament is a dosage form suitable for the subcutaneous, intradermal or intramuscular injection.

In a specific embodiment, the medicament is in a form of a depot formulation; and preferably, a long acting solid particle suspension injection.

In the fourth aspect, a method for preventing, treating or relieving pain is provided in the present invention, comprising administrating a therapeutically effective amount of the compound of formula (I) or solvates thereof of the first aspect, or the pharmaceutical composition of the second aspect, to a subject in need thereof.

In a preferred embodiment, the pain is a moderate to severe pain.

In a preferred embodiment, the moderate to severe pain is a postoperative pain. In a preferred embodiment, the postoperative pain is an acute or chronic postoperative pain.

It should be understood that, within the scope of the invention, the above technical features of the invention and the technical features described in detail hereinafter (such as Examples) can be combined with each other to form a new or preferred technical solution, which will not discuss herein one by one, due to the length of the description.

### DESCRIPTION OF DRAWINGS

FIG. 1 shows the concentration vs time curve of demethyl tramadol in rat plasma after different preparations of demethyl tramadol are administered.

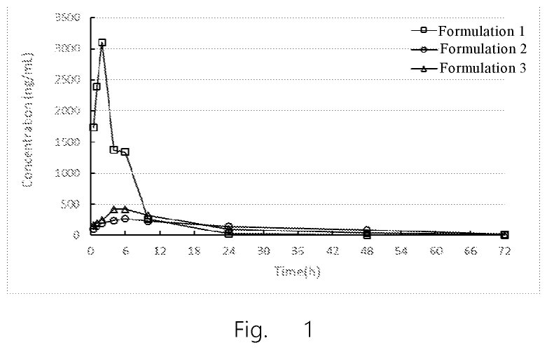

### MODES FOR CARRYING OUT THE INVENTION

After extensive and in-depth research, the inventor unexpectedly found that the preparation of O-demethyl tramadol into a specific palmitate, especially palmitate hemisalt, can significantly reduce the solubility of O-demethyl tramadol, thereby realizing the sustainable release of active O-demethyl tramadol. In addition, the specific O-demethyl tramadol palmitate of the present invention also exhibits excellent pharmacokinetic parameters, so that it can be safely and effectively administered. On this basis, the invention is completed.

The Compound and Pharmaceutical Composition of the Invention

As used herein, “compound of the invention”, “salt of the invention” and “palmitate of demethyl tramadol” have the same meaning, and can be used interchangeably. The above terms refer to the salt formed by O-demethyl tramadol and palmitic acid. As used herein, the term “demethyl tramadol” refers to O-demethyl tramadol. Tramadol is a low-risk opioid drug for moderate to severe pain. O-demethyl tramadol, as the active metabolite of tramadol, has higher activity than tramadol.

During the research, the inventors found that the solubility of O-demethyl tramadol was low, however, there were still various deficiencies. For example, O-demethyl tramadol is still insufficient to achieve the sustainable release of active demethyl tramadol, and other characteristics, especially pharmacokinetic parameters, such as Cₘₐₓ, are poor.

In order to obtain analgesic drugs that can exert their effects for a long time, in the prior art, it is commonly to prepare compounds with analgesic activities into low solubility salts, such as specific salt types. However, the technical effects achieved by preparing different compounds into different salt types are often inconsistent. The specific salt type applicable to some compounds may not be effective for other compounds. In addition, the solubility of some compounds can be decreased after they were prepared into a specific salt form, the pharmacokinetic parameters do not improve significantly.

In order to achieve the sustainable release of demethyl tramadol and improve its pharmacokinetic properties, the inventors prepared a salt formed by O-demethyl tramadol and palmitic acid. The solubility of this salt is further reduced, and its pharmacokinetic properties, such as Cₘₐₓ, are significantly improved. Therefore, this insoluble salt or its solvate can continuously release active drugs in vivo and maintain the effect concentration of the drug for a long time, thus not only achieving long-term analgesic effects, but also higher safety.

In a specific embodiment, the compound of the invention is the salt of formula (I) or a solvate thereof:

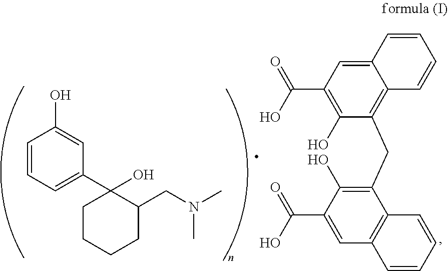

Wherein n is an integer of 1 to 4.

In a preferred embodiment, the compound of the invention is a semi-salt or monosalt of palmitate of demethyl tramadol; That is, n is 1 or 2; and preferably, 2.

It is known in the art that demethyl tramadol is a chiral compound, which has four stereoisomers, namely, levo demethyl tramadol, dextro demethyl tramadol and two racemic demethyl tramadol. According to the technical solution of the invention, there is no special restriction on the specific chiral structure of demethyl tramadol, that is, the racemate of demethyl tramadol (the mixture of levo demethyl tramadol and dextro demethyl tramadol with a molar ratio of 1:1) can be used, or the mixture of any one or more of levo demethyl tramadol and dextro demethyl tramadol in any proportion can be used. Among them, the chemical name of levo demethyl tramadol is (−)-(1S,2S)-2-[(N,N dimethylamino)methylene]-1-hydroxy-cyclohexyl phenol, and the chemical name of dextro demethyl tramadol is (+)-(1R,2R)-2-[(N,N dimethylamino)methylene]-1-hydroxy-cyclohexyl phenol.

The compound of the invention has excellent performance in many aspects. For example, in terms of hygroscopicity, the hygroscopicity of the compound of Formula (I) is not higher than 7%; preferably not higher than 4%; and more preferably not higher than 3%. The hygroscopicity can be detected by a conventional experiment known to a skilled person. For example, a sample of the compound of Formula (I) can be taken and placed in a dryer containing 80% RH saturated solution, balanced for 24 h, taken out and weighed as m₁; the sample is taken out and placed in a weighing bottle, with a thickness of about 1 mm, and weighed as m₂; and then the sample is put into a dryer with 80% RH saturated solution, balanced for 24 h, and weighed as m₃. After that, the hygroscopicity can be calculated according to the formula: hygroscopicity=(m₃−m₂)/(m₂−m₁)*100%.

In terms of light stability, the content of a single impurity of the compound of formula (I), after stored under a light condition (4500 lux±500 lux) for 10 days, is not higher than 0.1%; preferably, not higher than 0.06%; and more preferably, not higher than 0.04%; and the content of a single impurity, after stored under a light condition (4500 lux±500 lux) for 30 days, is not higher than 0.15%; preferably, not higher than 0.10%; and more preferably, not higher than 0.05%; and the content of total impurities is not higher than 0.40%; preferably, not higher than 0.20%; and more preferably, not higher than 0.05%.

In terms of thermal stability, the content of a single impurity of the compound of formula (I), after stored under a high temperature and high humidity condition (40° C., 75% humidity) for 10 days, is not higher than 0.08%; preferably, not higher than 0.06%; and more preferably, not higher than 0.05%; and the content of a single impurity, after stored under a high temperature and high humidity condition (40° C., 35 75% humidity) for 30 days, is not higher than 0.1%; preferably, not higher than 0.06%;

and more preferably, not higher than 0.05%; and the content of total impurities is not higher than 0.30%; preferably, not higher than 0.20%; and more preferably, not higher than 0.05%.

In addition to the above physical and chemical properties, the compound of the invention also shows excellent pharmacokinetic properties. For example, the plasma half-life (t_{1/2}) of the compound of formula (I) is at least 16 hours; and preferably, at least 17 hours. The maximum plasma concentration (Cₘₐₓ) of the compound of formula (I) is not higher than 500 ng/mL; and preferably not higher than 270 ng/mL.

In a specific embodiment, the compound of the invention can further form a solvate, such as a solvate formed by the compound of formula (I) and methanol, ethanol, n-propanol, isopropanol, ethyl acetate, acetone or water.

The compound of formula (I) of the invention or a solvate thereof can be in a form of solid particles with a D50 value of 0.1-100 μm.

Based on the compound of the invention, the invention further provides a pharmaceutical composition, which comprises a therapeutically effective amount of the above compound or a solvate thereof and pharmaceutically acceptable excipients.

The pharmaceutically acceptable excipients include one or more of the following excipients: suspending agent, surfactant, filler, preservative, isotonic regulator, pH regulator, buffer and water.

In a specific embodiment, the surfactant is selected from polysorbate 20 (Tween 20).

In a specific embodiment, the buffer is selected from sodium dihydrogen phosphate.

In a specific embodiment, the pharmaceutical composition is suitable for subcutaneous, intradermal or intramuscular injection. For example, the pharmaceutical composition is in a form of a long-acting preparation, preferably a long-acting solid particle suspension injection. 10 to 1000 mg; preferably 50 to 200 mg of the salt or a solate thereof is contained in 1 mL of the suspension injection.

The salt or a solvate thereof and pharmaceutical composition of the invention can be used to prevent or treat pain, such as preventing, treating or relieving pain by subcutaneous, intradermal or intramuscular injection. In a specific embodiment, the pain is a moderate to severe pain, such as postoperative pain. In a preferred embodiment, the postoperative pain is an acute or chronic postoperative pain.

The term “effective amount” or “therapeutically effective amount” used in the invention refers to the dosage or amount that can improve any parameter or clinical symptom. The actual dosage may vary with each patient and does not necessarily refer to the total amount that can eliminate all of the disease symptoms.

The term “not higher than” used in the invention refers to “less than” or “equal to” a number, which should be understood as including the number itself.

The term “D50” used in the invention refers to the particle size corresponding to a sample when the cumulative particle size distribution percentage reaches 50%. Its physical meaning is that the particle size of 50% of the particles are larger than it and the particle size of 50% of the particles are smaller than it. D50 is also named as median particle size or median particle size.

The terms used in the invention to describe approximate solubility are defined as follows (See Mingsheng Luo, Tianhui Gao, Complete List of Pharmaceutical Accessories [M]. Chengdu: Sichuan Science and Technology Press, 1982, P4): “soluble” means that 1 g (mL) of solute can be dissolved in 10˜less than 30 mL of solvent; “sparingly soluble” means that 1 g (mL) of solute can be dissolved in 30˜less than 100 mL of solvent; “slightly soluble” means that 1 g (mL) of solute can be dissolved in 100˜less than 1000 mL of solvent; “very slightly soluble” means that 1 g (mL) of solute can be dissolved in 1000˜less than 10000 mL of solvent.

Most of raw materials and reagents of the invention are commercially available or prepared according to known methods.

Advantages of the Invention:

1. The compound of the invention or a solvate thereof has excellent effects in many aspects, such as biological activity, safety, bioavailability, stability, solubility, etc;

2. The compound of the invention or a solvate has good stability, low hygroscopicity, and is extremely slightly soluble in water, which is conducive to the delayed release of drugs, and has good druggability; and

3. The invention provides a drug which can be produced through a simple process, and stably release local anesthetics in vivo for a long time. It can release local anesthetics for a long time, so that not only the analgesic effects on postoperative pain can be prolonged, but also be convenient for a doctor and patient to, thus having good drug compliance.

The invention will be further described in combination with specific embodiments. It should be understood that these embodiments are only used to explain the invention and not to limit the scope of the invention. The experimental methods without specific conditions specified in the following embodiments are usually based on the conventional conditions or the conditions recommended by the manufacturer. Unless otherwise stated, percentages and portions are calculated by weight.

### EXAMPLE

### Example 1

### Preparation of Di(Demethyl Tramadol) Palmitate

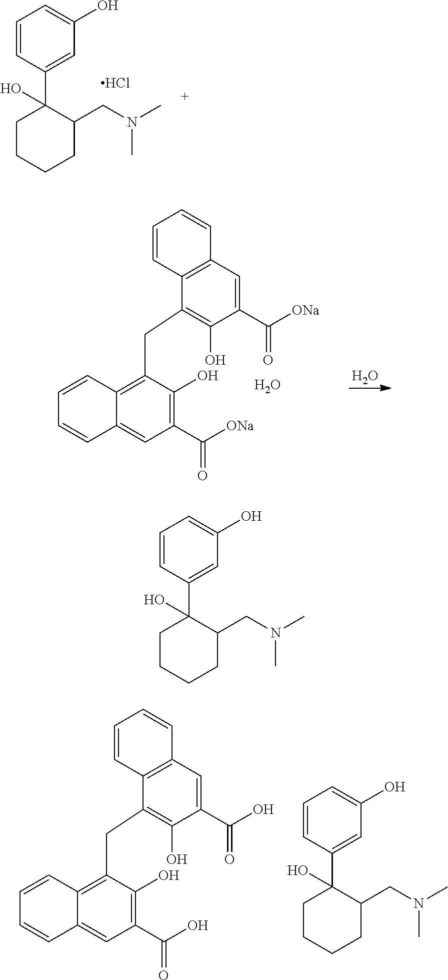

Racemic demethyl tramadol (28.5 g, 100 mol) and 86 mL of purified water were added into a 500 mL reaction flask, stirred and dissolved at room temperature. Disodium palmitate monohydrate (22.5 g, 50 mol) was dissolved in 270 mL of purified water and transferred into a constant pressure drip funnel, and added dropwise to the solution obtained in the previous step, during which a yellowish solid was precipitated. Upon addition (about 40 min), the reaction system was stirred at room temperature for 30 min and at 0° C. for 25 min. The filter cake was filtrated out, washed with 50 mL pre-cooled purified water, and vacuum-dried at 50° C. for 6 hr to yield 43.2 g of yellowish solids. The solids were transferred into a 500 mL eggplant shaped flask, 250 mL n-propanol was added, heated and stirred at 90° C. for crystallization for 4 hr, and then stirred overnight at room temperature. 41.2 g of light yellow solids were obtained by filtration and vacuum-drying at 60° C. for 5 hr with a yield of 93.2%. ¹-H NMR (400 MHz, DMSO-d6) δ 9.36 (brs, 2H), 8.27-8.08 (m, 2H), 7.73-7.55 (m, 1H), 7.22-7.07 (m, 2H), 7.05-6.84 (m, 3H), 6.67-6.60 (m, 1H), 5.03 (brs,1H), 4.68 (s, 1H), 2.92-2.78 (m, 1H), 2.72-2.26 (m, 1H), 2.54 (s, 3H), 2.52 (s, 3H), 2.20-2.06 (m, 1H), 1.94-1.29 (m, 8H).

### Example 2

### Demethyl Tramadol Palmitate Monosalt

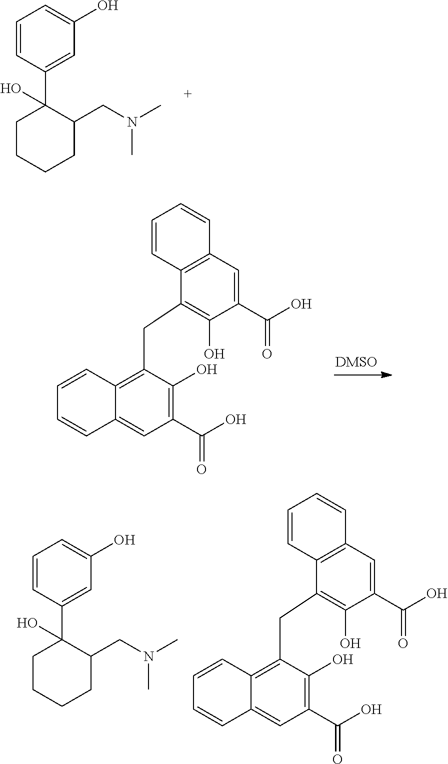

Palmitic acid (3.28 g, 8.46 mol) and DMSO (13 mL) were added into a 100 mL reaction flask, heated and dissolved. A racemic demethyl tramadol (2.11 g, 8.46 mol) solution in DMSO (6.3 mL) was added dropwise with stirring at room temperature, and stirred for another 30 min after addition. The reaction solution was added into 100 mL of purified water dropwise to precipitate yellow solids. The reaction system was stirred for 10 min at room temperature and 20 min at 0° C. The filter cake was filtrated out, washed with 10 mL pre-cooled purified water, and vacuum-dried at 60° C. for 4 hr to give 4.82 g of yellowish solids with a yield of 89.4%.

¹H NMR (400 MHz, DMSO-d6) δ 9.32 (brs, 1H), 8.91 (brs,1H), 8.30 (s, 2H), 8.22-8.06 (m, 1H), 7.85-7.62 (m, 1H), 7.36-7.01 (m, 5H), 6.99-6.80 (m, 2H), 6.67-6.57 (m, 1H), 5.04 (brs, 1H), 4.73 (s, 1H), 2.94-2.81 (m, 1H), 2.66 (s, 3H), 2.58-2.05 (m, 2H), 2.52 (s, 3H), 1.88-1.27 (m, 8H).

### Example 3

### Determination on Solubility of the Pharmaceutically Acceptable Salt of Demethyl tramadol

Determination Method:

1. chromatographic column: Agilent Pursuit C18, 3um, 4.6*150 mm

2. Mobile phase: 0.1% trifluoroacetic acid: acetonitrile=20: 80, flow rate: 1.0 mL/min, wavelength: 215 nm, column temperature: 35° C., injection volume: 10 μl;

3. Mobile phase preparation process:

1.0 mL of trifluoroacetic acid was added into 1000 mL of water, mixed well, and ultrasonicated;

4. Sample preparation process

About 10 mg of the sample to be tested was weighed and added into 1 mL of water, stirred for 24 hr, and centrifuged. The supernatant was taken as the sample for testing solubility. 10 mg of demethyl tramadol palmitate with the same structure as the sample to be tested was weighed and placed in a 100 mL measuring flask, 80% aqueous methanol solution was added to dissolve and dilute the demethyl tramadol palmitate to the scale, which was used as the corresponding reference solution.

According to the test, the solubility of the sample of Example 1 was 0.5 mg/mL, which can be converted into approximate solubility expressed as: 2000 mL of water is needed to disslove 1 g of the sample of Example 1. The solubility of the sample of Example 2 was 0.7 mg/mL, which can be converted into approximate solubility expressed as: 1428.6 mL of water is needed to dissolve 1 g of the sample of Example 2. The solubility of racemic demethyl tramadol hydrochloride is higher than 200 mg/mL, which can be converted into approximate solubility expressed as: 5 mL or less of water is needed to dissolve 1 g of racemic demethyl tramadol hydrochloride. The solubility of racemic demethyl tramadol acetate is higher than 50 mg/mL, which can be converted into approximate solubility expressed as: 20 mL or less of water is needed to dissolve 1 g of racemic demethyl tramadol acetate. The solubility of racemic demethyl tramadol free base is 1.4 mg/mL, which can be converted into approximate solubility expressed as: 714.3 mL of water is needed to dissolve 1 g of racemic demethyl tramadol free base.

Summing up, it can be seen that the palmitate of demethyl tramadol is very slightly soluble in water, which is conducive to the delayed release of the drug and has a good druggability.

### Example 4

### Determination on Hygroscopicity of the Pharmaceutically Acceptable Salt of Demethyl tramadol

Test process: a weighing bottle was taken, put into a dryer with 80% RH saturated solution, balanced for 24 h, taken out, and weighed as m₁; a sample was taken, spread into the weighing bottle with a thickness about 1 mm, and weighed as m₂; and then the weighing bottle was put into the dryer with 80% RH saturated solution, balanced for 24 h, and weighed as m₃.

Calculation formula: hygroscopicity=(m₃−m₂)/(m₂−m₁)*100%

Results: the hygroscopicity of the sample of Example 1 was 2.9%, and that of the sample of Example 2 was 6.3%.

### Example 5

### Stability Study

An appropriate amount of the sample of Example 1 and the sample of Example 2 of the invention were taken, placed in a glass plate under light conditions (4500 lux±500 lux) and high temperature and high humidity conditions (40° C., 75% humidity) for 30 days, respectively. Samples were taken at 0, 10, and 30 days, respectively to investigate the contents of a single impurity and total impurities of the three samples 25 under different conditions. Results can be found in Table 1 and Table 2.

The experiment conditions for purity determination are as follows:

1. Chromatographic column: Agilent Pursuit C18, 3 um, 4.6*150 mm;

2. Mobile phase: 0.1% trifluoroacetic acid (mobile phase A): acetonitrile (mobile phase B);

3. Flow rate: 1.0 ml/min, wavelength: 215 nm, column temperature: 35° C., injection volume: 10 μl;

and the column was eluted according to the following gradient:

| Time (min) | A % | B % |
|---|---|---|
| 0 | 90 | 10 |
| 20 | 15 | 85 |

TABLE 1
Results of stability investigation under light conditions

| | Day 0 | | Day 10 | | Day 30 | |
|---|---|---|---|---|---|---|
| | single | total | single | total | single | total |
| | impu- | impu- | impu- | impu- | impu- | impu- |
| | rity | rities | rity | rities | rity | rities |
| Sample of | ND | ND | 0.03% | 0.03% | 0.04% | 0.05% |
| Example 1 | | | | | | |
| Sample of | ND | ND | 0.09% | 0.20% | 0.14% | 0.37% |
| Example 2 | | | | | | |

TABLE 2
Results of stability investigation under high temperature
and high humidity conditions (40° C., 75% humidity)

| | Day 0 | | Day 10 | | Day 30 | |
|---|---|---|---|---|---|---|
| | single | total | single | total | single | total |
| | impu- | impu- | impu- | impu- | impu- | impu- |
| | rity | rities | rity | rities | rity | rities |
| Sample of | ND | ND | 0.04% | 0.04% | 0.04% | 0.04% |
| Example 1 | | | | | | |
| Sample of | ND | ND | 0.07% | 0.16% | 0.08% | 0.23% |
| Example 2 | | | | | | |

Note:
ND in the above Table 1 and Table 2 means that no impurities are detected.

From the above experimental results, it can be found that the palmitate of demethyl tramadol has good stability under light conditions as well as high temperature and high humidity conditions, which is conducive to a long-term storage. In particular, the stability of di(demethyl tramadol) palmitate is especially excellent under light conditions as well as high temperature and high humidity conditions.

### Example 6

### Formulation Preparation

The racemic demethyl tramadol, the sample of Example 1 (di(demethyl tramadol) palmitate) and the sample of Example 2 (demethyl tramadol palmitate monosalt) were prepared into a formulation, respectively, according to the composition shown in Table 3.

TABLE 3

| | Formulation 1 | Formulation 2 | Formulation 3 |
|---|---|---|---|
| Composition | Amount of the | Amount of the | Amount of the |
| | ingredient % | ingredient % | ingredient % |
| | (Weight volume | (Weight volume | (Weight volume |
| | ratio) | ratio) | ratio) |
| Raw matreial | 10% racemic | 17.20% | 25.58% |
| | demethyl | sample of | sample of |
| | tramadol | Example 1 | Example 2 |
| Tween 20 | 1.00% | 1.00% | 1.00% |
| NaH₂PO₄•H₂O | 0.14% | 0.14% | 0.14% |
| Water for | q.s. to 100% | q.s. to 100% | 0.14% |
| Injection | | | |

Preparation Method:

1) prescribed amounts of NaH₂PO₄⋅H₂O and Tween 20 were dissolved in water with a total weight of 80%, and pH was adjusted by 1 N NaOH solution to 7.00;

2) the demethyl tramadol/di(demethyl tramadol) palmitate/demethyl tramadol palmitate monosalt raw material as said above were added into the above system, and dispersed uniformly;

3) the above solution was sheared with a high shear machine for about 2 min;

4) The sheared solution was homogenized with a high-pressure homogenizer under a pressure of 400˜900 bar for 15 min to obtain a suspension.

### Example 7

### Study on Pharmacokinetics

1. Study on pharmacokinetics of demethyl tramadol/demethyl tramadol palmitate in SD rats after a single intramuscular injection

Animal and grades: SD rat, SPF grade

Sex and number: male, 15

15 Weight: 200˜220 g

Source: Zhejiang Weitong Lihua Laboratory Animal Technology Co., Ltd License No.: SCXK (Zhejiang) 2019-0001

Quality certificate No.: 2004030063

Assay process: 15 rats were randomly divided into 3 groups, and intramuscularly 20 injected with demethyl tramadol formulation, di(demethyl tramadol) palmitate formulation and demethyl tramadol palmitate monosalt formulation (prepared according to the method of Example 6 of the invention to obtain formulation 1, 2 and 3, respectively) at a dosage of 50 mg/kg (calculated by demethyl tramadol free base). After administration, blood samples were successively collected into K2EDTA anticoagulant tubes at about 0.5, 1, 2, 4, 6, 10, 24, 48 and 72 h, temporarily stored on ice, and centrifuged within 60 min (2-8° C., 8000 rpm for 5 min). Plasma was collected and transferred to centrifugation tubes, and stored at <-15° C.

Sample detection and data processing: a established LC-MS/MS method was used to detect plasma samples to obtain plasma concentration data and calculate pharmacokinetic parameters, including but not limited to Tmax, Cmax, AUC, etc. Detailed data are shown in the following table.

TABLE 4
Pharmacokinetic parameters of demethyl tramadol
in plasma of SD rats after administration

| | | Formulation | Formulation | Formulation |
|---|---|---|---|---|
| Parameters | Unit | 1 | 2 | 3 |
| t_{1/2} | h | 5.59 | 17.22 | 16.50 |
| Tₘₐₓ | h | 2.00 | 6.00 | 6.00 |
| Cₘₐₓ | ng/mL | 3162 | 268.7 | 480.76 |
| AUCₗₐₛₜ | h*ng/mL | 14129.56 | 8986.57 | 8347.28 |
| AUC_{inf} | h*ng/mL | 16525.64 | 9544.66 | 8930.59 |

Through the analysis in the above pharmacokinetic parameters of demethyl tramadol in the plasma of SD rats after administration, we found that the palmitate of demethyl tramadol has a longer half-life (t_{1/2}) compared with the free base of demethyl tramadol, which can be released for a long time, and has a good druggability for preparing a sustained release drug; and the maximum plasma concentration (Cₘₐₓ) is relatively low, so that drug sudden release and toxic side effects related to drug sudden release can be effectively avoided, and thus having better safety.

All the documents mentioned in the invention are cited as references in the application, as each document is cited separately as a reference. In addition, it should be understood that after reading the above teachings of the invention, those skilled in the art can make various changes or modifications to the invention, and these equivalent forms also fall within the scope of the claims attached to the application.

1. A salt of demethyl tramadol or a solvate thereof, wherein the salt of demethyl tramadol is a compound of formula (I) 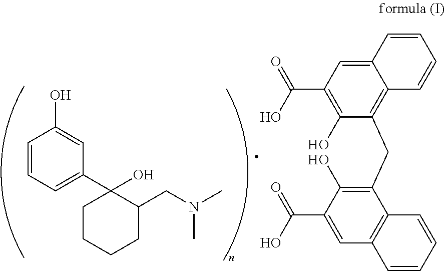
wherein n is an N is an integer from 1 to 4.

2. The salt of demethyl tramadol of claim 1 or a solvate thereof, wherein n is 1 or 2.

3. The salt of demethyl tramadol of claim 2 or a solvate thereof, wherein n is 2.

4. A pharmaceutical composition comprising the salt of demethyl tramadol of claim 1 or a solvate thereof, and optional pharmaceutically acceptable excipients.

5. The pharmaceutical composition of claim 4, wherein the pharmaceutical composition is a dosage form suitable for the subcutaneous, intradermal or intramuscular injection.

6. The pharmaceutical composition of claim 5, wherein the pharmaceutical composition is in a form of a depot formulation; and preferably, a long acting solid particle suspension injection.

7. (canceled)

8. (canceled)

9. (canceled)

10. A method for preventing, treating or relieving pain, comprising administrating a therapeutically effective amount of the salt of demethyl tramadol of claim 1 or a solvate thereof, or a pharmaceutical composition comprising the salt of demethyl tramadol of claim 1 or a solvate thereof, and optional pharmaceutically acceptable excipients, to a subject in need thereof.

11. The method of claim 10, wherein the salt of demethyl tramadol or the pharmaceutical composition is a dosage form suitable for the subcutaneous, intradermal or intramuscular injection.

12. The method of claim 11, wherein the salt of demethyl tramadol or the pharmaceutical composition in a form of a depot formulation; and preferably, a long acting solid particle suspension injection.

13. The method of claim 10, wherein the pain is a moderate to severe pain.

14. The method of claim 13, wherein the moderate to severe pain is a postoperative pain.

15. The method of claim 14, wherein the postoperative pain is an acute or chronic postoperative pain.
